SEQUENCE LISTING (30 sequences)

SEQ ID NO: 1 - DNA, 2004 bp, Homo sapiens
atggggagctccagactggcagccctgctcctgcctctcctcctcatagtcatcgacctctctgactctgctgggattggctttcgccacctgccccactggaacacccgctgtcctctggcctcccacacggatgacagtttcactggaagttctgcctatatcccttgccgcacctggtgggccctcttctccacaaagccttggtgtgtgcgagtctggcactgttcccgctgtttgtgccagcatctgctgtcaggtggctcaggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctggtgtggcggtcagatgtccagtttgcctggaagcacctcttgtgtccggatgtctcttacagacacctggggctcttgatcctggcactgctggccctcctcaccctactgggtgttgttctggccctcacctgccggcgcccacagtcaggcccgggcccagcgcggccagtgctcctcctgcacgcggcggactcggaggcgcagcggcgcctggtgggagcgctggctgaactgctacgggcagcgctgggcggcgggcgcgacgtgatcgtggacctgtgggaggggaggcacgtggcgcgcgtgggcccgctgccgtggctctgggcggcgcggacgcgcgtagcgcgggagcagggcactgtgctgctgctgtggagcggcgccgaccttcgcccggtcagcggccccgacccccgcgccgcgcccctgctcgccctgctccacgctgccccgcgcccgctgctgctgctcgcttacttcagtcgcctctgcgccaagggcgacatccccccgccgctgcgcgccctgccgcgctaccgcctgctgcgcgacctgccgcgtctgctgcgggcgctggacgcgcggcctttcgcagaggccaccagctggggccgccttggggcgcggcagcgcaggcagagccgcctagagctgtgcagccggctcgaacgagaggccgcccgacttgcagacctaggttga

SEQ ID NO: 2 - DNA, 1602 bp, Homo sapiens
atggggagctccagactggcagccctgctcctgcctctcctcctcatagtcatcgacctctctgactctgctgggattggctttcgccacctgccccactggaacacccgctgtcctctggcctcccacacggatgacagtttcactggaagttctgcctatatcccttgccgcacctggtgggccctcttctccacaaagccttggtgtgtgcgagtctggcactgttcccgctgtttgtgccagcatctgctgtcaggtggctcaggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacagctcgagagtcagatgggtggtatgttttggagaaggtggacctgcacccccagctctgcttcaagttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggcccggggctcaagcccagtgtcactagacctcatcattcccttcctgaggccagggtgctgtgtcctgctccatgcttcactcagctccccgggaggagaagatgcctggctcataggggtggggggctctgtgccctcaggtgtggcggtcagatgtccagtttgcctggaagcacctcttgtgtccggatgtctcttacagacacctggggctcttgatcctggcactgctggccctcctcaccctactgggtgttgttctggccctcacctgccggcgcccacagtcaggcccgggcccagcgcggccagtgctcctcctgcacgcggcggactcggaggcgcagcggcgcctggtgggagcgctggctga

SEQ ID NO: 3 - protein, 667 aa, Homo sapiens
MGSSRLAALLLPLLLIVIDLSDSAGIGFRHLPHWNTRCPLASHTDDSFTGSSAYIPCRTWWALFSTKPWCVRVWHCSRCLCQHLLSGGSGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRHLGLLILALLALLTLLGVVLALTCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 4 - protein, 533 aa, Homo sapiens
MGSSRLAALLLPLLLIVIDLSDSAGIGFRHLPHWNTRCPLASHTDDSFTGSSAYIPCRTWWALFSTKPWCVRVWHCSRCLCQHLLSGGSGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLLHASLSSPGGEDAWLIGVGGSVPSGVAVRCPVCLEAPLVSGCLLQTPGALDPGTAGPPHPTGCCSGPHLPAPTVRPGPSAASAPPARGGLGGAAAPGGSAG

SEQ ID NO: 5 - protein, 191 aa, Artificial Sequence
  Synthetic Construct
EMGSSRLAALLLPLLLIVIDLSDSAGIGFRHLPHWNTRCPLASHTRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEMARTSGSQCTSLTTASTLRWSWP

SEQ ID NO: 6 - protein, 848 aa, Artificial Sequence
  Synthetic Construct
EMGSSRLAALLLPLLLIVIDLSDSAGIGFRHLPHWNTRCPLASHTVFNGASSTSWCRNPKSLPHSSSIGDTRCQHLLRGSCCLVVTCLRRAITFPSPPQTSPTRDFALKGPNLRIQRHGKVFPDWTHKGMEDPSSPLICCLRPGLFGEMLAPFLLLLTHSRSPLSFRPCSHVPGALIAQKPMGSSRLAALLLPLLLIVIDLSDSAGIGFRHLPHWNTRCPLASHTDDSFTGSSAYIPCRTWWALFSTKPWCVRVWHCSRCLCQHLLSGGSGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRHLGLLILALLALLTLLGVVLALTCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 7 - protein, 701 aa, Artificial Sequence
  Synthetic Construct
EMLAPFLLLLTHSRSPLSFRPCSHVPGALIAQKPMGSSRLAALLLPLLLIVIDLSDSAGIGFRHLPHWNTRCPLASHTDDSFTGSSAYIPCRTWWALFSTKPWCVRVWHCSRCLCQHLLSGGSGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRHLGLLILALLALLTLLGVVLALTCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 8 - protein, 174 aa, Artificial Sequence
  Synthetic Construct
EMGSSRLAALLLPLLLIVIDLSDSAGIGFRHLPHWNTRCPLASHTDDSFTGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECE

SEQ ID NO: 9 - protein, 187 aa, Artificial Sequence
  Synthetic Construct
EMGSSRLAALLLPLLLIVIDLSDSAGIGFRHLPHWNTRCPLASHTEVLPISLAAPGGPSSPQSLGVCESGTVPAVCASICCQVAQVFNGASSTSWCRNPKSLPHSSSIGDTRCQHLLRGSCCLVVTCLRRAITFPSPPQTSPTRDFALKGPNLRIQRHGKVFPDWTHKGMEDPSSPLICCLRPGLFG

SEQ ID NO: 10 - DNA, 810 bp, Homo sapiens
atggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaa

SEQ ID NO: 11 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 12 - protein, 125 aa, Artificial Sequence
  Synthetic Construct
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCT

SEQ ID NO: 13 - protein, 143 aa, Artificial Sequence
  Synthetic Construct
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELK

SEQ ID NO: 14 - protein, 111 aa, Artificial Sequence
  Synthetic Construct
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNE

SEQ ID NO: 15 - DNA, 459 bp, Homo sapiens
atgagccgcctgcccgtcctgctcctgctccaactcctggtccgccccggactccaagctcccatgacccagacaacgcccttgaagacaagctgggttaactgctctaacatgatcgatgaaattataacacacttaaagcagccacctttgcctttgctggacttcaacaacctcaatggggaagaccaagacattctgatggaaaataaccttcgaaggccaaacctggaggcattcaacagggctgtcaagagtttacagaacgcatcagcaattgagagcattcttaaaaatctcctgccatgtctgcccctggccacggccgcacccacgcgacatccaatccatatcaaggacggtgactggaatgaattccggaggaaactgacgttctatctgaaaacccttgagaatgcgcaggctcaacagacgactttgagcctcgcgatcttttga

SEQ ID NO: 16 - protein, 152 aa, Homo sapiens
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 17 - DNA, 1380 bp, Homo sapiens
atgacaattctaggtacaacttttggcatggttttttctttacttcaagtcgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggggagatggatcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtga

SEQ ID NO: 18 - protein, 459 aa, Homo sapiens
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDPILLTISILSFFSVALLVILACVLWKKRIKPIVWPSLPDHKKTLEHLCKKPRKNLNVSFNPESFLDCQIHRVDDIQARDEVEGFLQDTFPQQLEESEKQRLGGDVQSPNCPSEDVVITPESFGRDSSLTCLAGNVSACDAPILSSSRSLDCRESGKNGPHVYQDLLLSLGTTNSTLPPPFSLQSGILTLNPVAQGQPILTSLGSNQEEAYVTMSSFYQNQ

SEQ ID NO: 19 - protein, 459 aa, Artificial Sequence; identical to SEQ ID NO: 18
  Synthetic Construct

SEQ ID NO: 20 - protein, 52 aa, Artificial Sequence
  Synthetic Construct
MTILGTTFGMVFSLLQVVSGESGYAQNGDLEDAELDDYSFSCYSQLEVNGSQ

SEQ ID NO: 21 - protein, 180 aa, Artificial Sequence
  Synthetic Construct
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTD

SEQ ID NO: 22 - protein, 150 aa, Artificial Sequence
  Synthetic Construct
MTILGTTFGMVFSLLQVVSGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIGKKLYIKVWLSLLGYLKTLCLDIL

SEQ ID NO: 23 - protein, 59 aa, Artificial Sequence
  Synthetic Construct
MTILGTTFGMVFSLLQVVSGESGYAQNGDLEDAELDDYSFSCYSQLEVNGSQHSLTCAF

SEQ ID NO: 24 - protein, 65 aa, Artificial Sequence
  Synthetic Construct
AAMYEIKVRSIPDHYFKGFWSEWSPSYYFRTPEINNSSGLSLSYGPVSPIIRRLWNIFVRNQEKI

SEQ ID NO: 25 - DNA, 1029 bp, Homo sapiens
atgcctcagacttgtgatgggactgggcagatgcatctgggaagtaactgctgcaagaacggacagacactgctgcagagaacttgccacggtgtttcatgctgtggctggtggttccaggctgcacgctccattctaggaaaggggccctcagcccagtcccttgcaggctggaccttggagagtgaggccctgaggcgagacatgggcacctggctcctggcctgcatctgcatctgcacctgtgtctgcttgggagtctctgtcacaggggaaggacaagggccaaggtctagaaccttcacctgcctcaccaacaacattctcaggatcgattgccactggtctgccccagagctgggacagggctccagcccctggctcctcttcaccaggctcctggcggcacacataagtgcatcttgcggggcagtgagtgcaccgtcgtgctgccacctgaggcagtgctcgtgccatctgacaatttcaccatcactttccaccactgcatgtctgggagggagcaggtcagcctggtggacccggagtacctgccccggagacacgagcaacatcagttctggccactgcatcctgacctggagcatcagtcctgccttggagccaatgaccacacttctcagctatgagctggccttcaagaagcaggaagaggcctgggagcaggcccagcacagggatcacattgtcggggtgacctggcttatacttgaagcctttgagctggaccctggctttatccatgaggccaggctgcgtgtccagatggccacactggaggatgatgtggtagaggaggagcgttatacaggccagtggagtgagtggagccagcctgtgtgcttccaggctccccagagacaaggccctctgatcccaccctgggggtggccaggcaacacccttgttgctgtgtccatctttctcctgctgactggcccgacctacctcctgttcaagctgtcgcccagacttggatgggggcccacggggccggtgtgctgttga

SEQ ID NO: 26 - DNA, 1566 bp, Homo sapiens
atgggactgggcagatgcatctgggaaggctggaccttggagagtgaggccctgaggcgagacatgggcacctggctcctggcctgcatctgcatctgcacctgtgtctgcttgggagtctctgtcacaggggaaggacaagggccaaggtctagaaccttcacctgcctcaccaacaacattctcaggatcgattgccactggtctgccccagagctgggacagggctccagcccctggctcctcttcaccagcaaccaggctcctggcggcacacataagtgcatcttgcggggcagtgagtgcaccgtcgtgctgccacctgaggcagtgctcgtgccatctgacaatttcaccatcactttccaccactgcatgtctgggagggagcaggtcagcctggtggacccggagtacctgccccggagacacgttaagctggacccgccctctgacttgcagagcaacatcagttctggccactgcatcctgacctggagcatcagtcctgccttggagccaatgaccacacttctcagctatgagctggccttcaagaagcaggaagaggcctgggagcaggcccagcacagggatcacattgtcggggtgacctggcttatacttgaagcctttgagctggaccctggctttatccatgaggccaggctgcgtgtccagatggccacactggaggatgatgtggtagaggaggagcgttatacaggccagtggagtgagtggagccagcctgtgtgcttccaggctccccagagacaaggccctctgatcccaccctgggggtggccaggcaacacccttgttgctgtgtccatctttctcctgctgactggcccgacctacctcctgttcaagctgtcgcccagggtgaagagaatcttctaccagaacgtgccctctccagcgatgttcttccagcccctctacagtgtacacaatgggaacttccagacttggatgggggcccacggggccggtgtgctgttgagccaggactgtgctggcaccccacagggagccttggagccctgcgtccaggaggccactgcactgctcacttgtggcccagcgcgtccttggaaatctgtggccctggaggaggaacaggagggccctgggaccaggctcccggggaacctgagctcagaggatgtgctgccagcagggtgtacggagtggagggtacagacgcttgcctatctgccacaggaggactgggcccccacgtccctgactaggccggctcccccagactcagagggcagcaggagcagcagcagcagcagcagcagcaacaacaacaactactgtgccttgggctgctatgggggatggcacctctcagccctcccaggaaacacacagagctctgggcccatcccagccctggcctgtggcctttcttgtgaccatcagggcctggagacccagcaaggagttgcctgggtgctggctggtcactgccagaggcctgggctgcatgaggacctccagggcatgttgctcccttctgtcctcagcaaggctcggtcctggacattctag

SEQ ID NO: 27 - protein, 342 aa, Homo sapiens
MPQTCDGTGQMHLGSNCCKNGQTLLQRTCHGVSCCGWWFQAARSILGKGPSAQSLAGWTLESEALRRDMGTWLLACICICTCVCLGVSVTGEGQGPRSRTFTCLTNNILRIDCHWSAPELGQGSSPWLLFTRLLAAHISASCGAVSAPSCCHLRQCSCHLTISPSLSTTACLGGSRSAWWTRSTCPGDTSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWPGNTLVAVSIFLLLTGPTYLLFKLSPRLGWGPTGPVCC

SEQ ID NO: 28 - protein, 521 aa, Homo sapiens
MGLGRCIWEGWTLESEALRRDMGTWLLACICICTCVCLGVSVTGEGQGPRSRTFTCLTNNILRIDCHWSAPELGQGSSPWLLFTSNQAPGGTHKCILRGSECTVVLPPEAVLVPSDNFTITFHHCMSGREQVSLVDPEYLPRRHVKLDPPSDLQSNISSGHCILTWSISPALEPMTTLLSYELAFKKQEEAWEQAQHRDHIVGVTWLILEAFELDPGFIHEARLRVQMATLEDDVVEEERYTGQWSEWSQPVCFQAPQRQGPLIPPWGWPGNTLVAVSIFLLLTGPTYLLFKLSPRVKRIFYQNVPSPAMFFQPLYSVHNGNFQTWMGAHGAGVLLSQDCAGTPQGALEPCVQEATALLTCGPARPWKSVALEEEQEGPGTRLPGNLSSEDVLPAGCTEWRVQTLAYLPQEDWAPTSLTRPAPPDSEGSRSSSSSSSSNNNNYCALGCYGGWHLSALPGNTQSSGPIPALACGLSCDHQGLETQQGVAWVLAGHCQRPGLHEDLQGMLLPSVLSKARSWTF

SEQ ID NO: 29 - DNA, 690 bp, Homo sapiens
atgaccccgcagcttctcctggcccttgtcctctgggccagctgcccgccctgcagtggaaggaaagggcccccagcagctctgacactgccccgggtgcaatgccgagcctctcggtacccgatcgccgtggattgctcctggaccctgccgcctgctccaaactccaccagccccgtgtccttcattgccacgtacaggctcggcatggctgcccggggccacagctggccctgcctgcagcagacgccaacgtccaccagctgcaccatcacggatgtccagctgttctccatggctccctacgtgctcaatgtcaccgccgtccacccctggggctccagcagcagcttcgtgcctttcataacagagcacatcatcaagcccgaccctccagaaggcgtgcgcctaagccccctcgctgagcgccagctacaggtgcagtgggagcctcccgggtcctggcccttcccagagatcttctcactgaagtactggatccgttacaagcgtcagggagctgcgcgcttccaccgggtggggcccattgaagccacgtccttcatcctcagggctgtgcggccccgagccaggtactacgtccaagtggcggctcaggacctcacagactacggggaactgagtgactggagtctccccgccactgccacaatgagcctgggcaagtag

SEQ ID NO: 30 - protein, 210 aa, Homo sapiens
MTPQLLLALVLWASCPPCSGRKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTD

(19) United States
(12) Patent Application Publication
(10) Pub. No.: US 2023/0313310 A1
(43) Pub. Date: Oct. 5, 2023

(54) PREDICTION OF RADIOTHERAPY RESPONSE FOR PROSTATE CANCER SUBJECT BASED ON INTERLEUKIN GENES

(71) Applicant: KONINKLIJKE PHILIPS N.V., EINDHOVEN (NL)

(72) Inventors: Ralf Dieter Hoffmann, Brueggen (DE); Joukje Garrelina Orsel, Valkenswaard (NL); Ron Martinus Laurentius van Lieshout, Geldrop (NL); Maud de Klerk-Starmans, Eindhoven (NL)

(21) Appl. No.: 17/908,619

(22) PCT Filed: Mar. 4, 2021
(86) PCT No.: PCT/EP2021/055424

§ 371 Date: Sep. 1, 2022

(30) Foreign Application Priority Data
Mar. 5, 2020 (EP) 20161129.0

(51) Int. Cl. C12Q 1/6886; G16B 25/10; G16B 40/00

(52) U.S. Cl. CPC C12Q 1/6886; G16B 25/10; G16B 40/00; C12Q 2600/106; C12Q 2600/158

(57) ABSTRACT

The invention relates to a method of predicting a response of a prostate cancer subject to radiotherapy, comprising determining or receiving the result of a determination of a gene expression profile for each of three or more interleukin genes selected from the group consisting of: IL17RE, IL1B, IL3, IL7R, IL9R, and EBI3, said gene expression profiles being determined in a biological sample obtained from the subject, and determining, preferably by a processor, the prediction of the radiotherapy response based on the gene expression profiles for the three or more interleukin genes.

### FIELD OF THE INVENTION

The invention relates to a method of predicting a response of a prostate cancer subject to radiotherapy. Moreover, the invention relates to a diagnostic kit, to a use of the kit in a method of predicting a response of a prostate cancer subject to radiotherapy, to a use of a gene expression profile for each of three or more interleukin genes in radiotherapy prediction for a prostate cancer subject, and to a corresponding computer program product.

### BACKGROUND OF THE INVENTION

Cancer is a class of diseases in which a group of cells displays uncontrolled growth, invasion and sometimes metastasis. These three malignant properties of cancers differentiate them from benign tumours, which are self-limited and do not invade or metastasize. Prostate Cancer (PCa) is the second most commonly-occurring non-skin malignancy in men, with an estimated 1.3 million new cases diagnosed and 360,000 deaths world-wide in 2018 (see Bray F. et al., “Global cancer statistics 2018: GLOBOCAN estimates of incidence and mortality worldwide for 36 cancers in 185 countries”, CA Cancer J Clin, Vol. 68, No. 6, pages 394-424, 2018). In the US, about 90% of the new cases concern localized cancer, meaning that metastases have not yet been formed (see ACS (American Cancer Society), “Cancer Facts & FIGS. 2010”, 2010).

For the treatment of primary localized prostate cancer, several radical therapies are available, of which surgery (radical prostatectomy, RP) and radiation therapy (RT) are most commonly used. RT is administered via an external beam or via the implantation of radioactive seeds into the prostate (brachytherapy) or a combination of both. It is especially preferable for patients who are not eligible for surgery or have been diagnosed with a tumour in an advanced localized or regional stage. Radical RT is provided to up to 50% of patients diagnosed with localized prostate cancer in the US (see ACS, 2010, ibid).

After treatment, prostate cancer antigen (PSA) levels in the blood are measured for disease monitoring. An increase of the blood PSA level provides a biochemical surrogate measure for cancer recurrence or progression. However, the variation in reported biochemical progression-free survival (bPFS) is large (see Grimm P. et al., “Comparative analysis of prostate-specific antigen free survival outcomes for patients with low, intermediate and high risk prostate cancer treatment by radical therapy. Results from the Prostate Cancer Results Study Group”, BJU Int, Suppl. 1, pages 22-29, 2012). For many patients, the bPFS at 5 or even 10 years after radical RT may lie above 90%. Unfortunately, for the group of patients at medium and especially higher risk of recurrence, the bPFS can drop to around 40% at 5 years, depending on the type of RT used (see Grimm P. et al., 2012, ibid).

A large number of the patients with primary localized prostate cancer that are not treated with RT will undergo RP (see ACS, 2010, ibid). After RP, an average of 60% of patients in the highest risk group experience biochemical recurrence after 5 and 10 years (see Grimm P. et al., 2012, ibid). In case of biochemical progression after RP, one of the main challenges is the uncertainty whether this is due to recurring localized disease, one or more metastases or even an indolent disease that will not lead to clinical disease progression (see Dal Pra A. et al., “Contemporary role of postoperative radiotherapy for prostate cancer”, Transl Androl Urol, Vo. 7, No. 3, pages 399-413, 2018, and Herrera F. G. and Berthold D. R., “Radiation therapy after radical prostatectomy: Implications for clinicians”, Front Oncol, Vol. 6, No. 117, 2016). RT to eradicate remaining cancer cells in the prostate bed is one of the main treatment options to salvage survival after a PSA increase following RP. The effectiveness of salvage radiotherapy (SRT) results in 5-year bPFS for 18% to 90% of patients, depending on multiple factors (see Herrera F. G. and Berthold D. R., 2016, ibid, and Pisansky T. M. et al., “Salvage radiation therapy dose response for biochemical failure of prostate cancer after prostatectomy—A multi-institutional observational study”, Int J Radiat Oncol Biol Phys, Vol. 96, No. 5, pages 1046-1053, 2016).

It is clear that for certain patient groups, radical or salvage RT is not effective. Their situation is even worsened by the serious side effects that RT can cause, such as bowel inflammation and dysfunction, urinary incontinence and erectile dysfunction (see Resnick M. J. et al., “Long-term functional outcomes after treatment for localized prostate cancer”, N Engl J Med, Vol. 368, No. 5, pages 436-445, 2013, and Hegarty S. E. et al., “Radiation therapy after radical prostatectomy for prostate cancer: Evaluation of complications and influence of radiation timing on outcomes in a large, population-based cohort”, PLoS One, Vol. 10, No. 2, 2015). In addition, the median cost of one course of RT based on Medicare reimbursement is $18,000, with a wide variation up to about $40,000 (see Paravati A. J. et al., “Variation in the cost of radiation therapy among medicare patients with cancer”, J Oncol Pract, Vol. 11, No. 5, pages 403-409, 2015). These figures do not include the considerable longitudinal costs of follow-up care after radical and salvage RT.

An improved prediction of effectiveness of RT for each patient, be it in the radical or the salvage setting, would improve therapy selection and potentially survival. This can be achieved by 1) optimizing RT for those patients where RT is predicted to be effective (e.g., by dose escalation or a different starting time) and 2) guiding patients where RT is predicted not to be effective to an alternative, potentially more effective form of treatment. Further, this would reduce suffering for those patients who would be spared ineffective therapy and would reduce costs spent on ineffective therapies.

Numerous investigations have been conducted into measures for response prediction of radical RT (see Hall W. A. et al., “Biomarkers of outcome in patients with localized prostate cancer treated with radiotherapy”, Semin Radiat Oncol, Vol. 27, pages 11-20, 2016, and Raymond E. et al., “An appraisal of analytical tools used in predicting clinical outcomes following radiation therapy treatment of men with prostate cancer: A systematic review”, Radiat Oncol, Vol. 12, No. 1, page 56, 2017) and SRT (see Herrera F. G. and Berthold D. R., 2016, ibid). Many of these measures depend on the concentration of the blood-based biomarker PSA. Metrics investigated for prediction of response before start of RT (radical as well as salvage) include the absolute value of the PSA concentration, its absolute value relative to the prostate volume, the absolute increase over a certain time and the doubling time. Other frequently considered factors are the Gleason score and the clinical tumour stage. For the SRT setting, additional factors are relevant, e.g., surgical margin status, time to recurrence after RP, pre-/peri-surgical PSA values and clinico-pathological parameters.

Although these clinical variables provide limited improvements in patient stratification in various risk groups, there is a need for better predictive tools.

A wide range of biomarker candidates in tissue and bodily fluids has been investigated, but validation is often limited and generally demonstrates prognostic information and not a predictive (therapy-specific) value (see Hall W. A. et al., 2016, ibid). A small number of gene expression panels is currently being validated by commercial organizations. One or a few of these may show predictive value for RT in future (see Dal Pra A. et al., 2018, ibid).

In conclusion, a strong need for better prediction of response to RT remains, for primary prostate cancer as well as for the post-surgery setting.

WO 2018/039490 A1 discloses methods, compositions, and kits for identifying individuals who will be responsive to post-operative radiation therapy for treatment of prostate cancer are disclosed. In particular, the document relates to a genomic signature based on expression levels of DNA Damage Repair genes that can be used to identify individuals likely to benefit from post-operative radiation therapy after a prostatectomy.

Zhao S. G. et al., “Development and validation of a 24-gene predictor of response to postoperative radiotherapy in prostate cancer: a matched, retrospective analysis”, The Lancet Oncology, Vol. 17, No. 11, pages 1612-1620, 2016, starts out from the premise that postoperative radiotherapy has an important role in the treatment of prostate cancer, but that personalised patient selection could improve outcomes and spare unnecessary toxicity. The document aims at developing and validating a gene expression signature to predict which patients would benefit most from postoperative radiotherapy.

### SUMMARY OF THE INVENTION

It is an objective of the invention to provide a method of predicting a response of a prostate cancer subject to radiotherapy, which allows to make better treatment decisions. It is a further objective of the invention to provide a diagnostic kit, a use of the kit in a method of predicting a response of a prostate cancer subject to radiotherapy, a use of a gene expression profile for each of three or more interleukin genes in radiotherapy prediction for a prostate cancer subject, and a corresponding computer program product.

In a first aspect of the present invention, a method of predicting a response of a prostate cancer subject to radiotherapy is presented, comprising:
determining or receiving the result of a determination of a gene expression profile for each of three or more, for example, 3, 4, 5 or all, interleukin genes selected from the group consisting of: IL17RE, IL1B, IL3, IL7R, IL9R, and EBI3, said gene expression profiles being determined in a biological sample obtained from the subject,
determining, preferably by a processor, the prediction of the radiotherapy response based on the gene expression profiles for the three or more interleukin genes, and
optionally, providing the prediction or a therapy recommendation based on the prediction to a medical caregiver or the subject.

In recent years, the importance of the immune system in cancer inhibition as well as in cancer initiation, promotion and metastasis has become very evident (see Mantovani A. et al., “Cancer-related inflammation”, Nature, Vol. 454, No. 7203, pages 436-444, 2008, and Giraldo N. A. et al., “The clinical role of the TME in solid cancer”, Br J Cancer, Vol. 120, No. 1, pages 45-53, 2019). The immune cells and the molecules they secrete form a crucial part of the tumour microenvironment and most immune cells can infiltrate the tumour tissue. The immune system and the tumour affect and shape one another. Thus, anti-tumour immunity can prevent tumour formation while an inflammatory tumour environment may promote cancer initiation and proliferation. At the same time, tumour cells that may have originated in an immune system-independent manner will shape the immune microenvironment by recruiting immune cells and can have a pro-inflammatory effect while also suppressing anti-cancer immunity.

Some of the immune cells in the tumour microenvironment will have either a general tumour-promoting or a general tumour-inhibiting effect, while other immune cells exhibit plasticity and show both tumour-promoting and tumour-inhibiting potential. Thus, the overall immune microenvironment of the tumour is a mixture of the various immune cells present, the cytokines they produce and their interactions with tumour cells and with other cells in the tumour microenvironment (see Giraldo N. A. et al., 2019, ibid).

The principles described above with regard to the role of the immune system in cancer in general also apply to prostate cancer. Chronic inflammation has been linked to the formation of benign as well as malignant prostate tissue (see Hall W. A. et al., 2016, ibid) and most prostate cancer tissue samples show immune cell infiltrates. The presence of specific immune cells with a pro-tumour effect has been correlated with worse prognosis, while tumours in which natural killer cells were more activated showed better response to therapy and longer recurrence-free periods (see Shiao S. L. et al., “Regulation of prostate cancer progression by tumor microenvironment”, Cancer Lett, Vol. 380, No. 1, pages 340-348, 2016).

While a therapy will be influenced by the immune components of the tumour microenvironment, RT itself extensively affects the make-up of these components (see Barker H. E. et al., “The tumor microenvironment after radiotherapy: Mechanisms of resistance or recurrence”, Nat Rev Cancer, Vol. 15, No. 7, pages 409-425, 2015). Because suppressive cell types are comparably radiation-insensitive, their relative numbers will increase. Counteractively, the inflicted radiation damage activates cell survival pathways and stimulates the immune system, triggering inflammatory responses and immune cell recruitment. Whether the net effect will be tumour-promoting or tumour-suppressing is as yet uncertain, but its potential for enhancement of cancer immunotherapies is being investigated.

The present invention is based on the idea that, since the status of the immune system and of the immune microenvironment have an impact on therapy effectiveness, the ability to identify markers predictive for this effect might help to be better able to predict overall RT response.

Interleukins are one of the main groups of cytokines. They form a large family of over 50 molecules that play a central role in the regulation of the immune system (see Brocker C. et al., “Evolutionary divergence and functions of the human interleukin (IL) gene family”, Hum Genomics, Vol. 5, No. 1, pages 30-55, 2010). Based on their sequence, interleukins can be clustered in 4 major groups, but overall their sequence similarity is relatively weak. The main function of these proteins is to modulate growth, differentiation and activation during an immune response. Multiple family members are involved in the activation or suppression of T cells, which in their turn may play a role in, for example, clearing of tumour cells or the modulation of inflammatory responses after RT.

Interleukins exert their function by enabling communication between cells. They are secreted by immune cells and reach their target cells via interstitial fluid and the blood circulation. The target cells express interleukin receptor molecules on their surface to which the interleukins can bind, thereby activating or inhibiting a signalling cascade inside the cell. The signalling cascade finally influences the expression of proteins resulting in immune cell growth, differentiation and activation.

Which function interleukins exactly perform depends on the secreting cells, the target cells and the phase of the immune response. One interleukin can bind to multiple different receptors. Moreover, an interleukin can have both pro- and anti-inflammatory effects. This highly complicates the determination of the precise functions of each interleukin. Therefore, it is extremely difficult to conclude based on literature which members of the interleukin family or their receptors might be specifically predictive for the response of prostate cancer patients to radical RT or SRT.

Several investigations have been performed as to the change in levels of one or more interleukins during RT for prostate cancer, or even therapy targeting an interleukin. Very recently, a report was made on the investigation of a number of interleukins in serum for the prediction of RT response in prostate cancer (see Hall W. A. et al., “The influence of the pretreatment host immune inflammatory state and response to radiation therapy in high-risk adenocarcinoma of the prostate: A validation study from NRG Oncology/RTOG 0521”), Vol. 102, No. 3, pages S13-S14, 2018). A link with disease free survival was found for the level of IL10 in serum. A few other interleukins were found to be linked not to survival but to side effects of radiation. This highlights the need for a complete overview of multiple IL components and their activity.

The identified interleukin genes IL17RE, IL1B, IL3, IL7R, IL9R, and EBI3 were identified as follows: A group of 538 prostate cancer patients were treated with RP and the prostate cancer tissue was stored. A number of these patients experienced biochemical recurrence and was treated with SRT. For 151 of these patients, the RNA expressed in the originally stored prostate cancer tissue was analysed using RNA sequencing. The mRNA expression of interleukins and their receptors was compared for the 26 out of 151 patients that died due to prostate cancer, versus the 125 out of 151 patients who survived. For the six molecules IL17RE, IL1B, IL3, IL7R, IL9R, and EBI3, the expression level was significantly different for the survivors, suggesting that they have value in the prediction of survival after SRT. Several of these six molecules were differentially expressed in other data sets as well, as described in more detail further below.

The term “IL17RE” refers to the human Interleukin 17 Receptor E gene (Ensembl: ENSG00000163701), for example, to the sequence as defined in NCBI Reference Sequence NM_153480.2 or in NCBI Reference Sequence NM_001193380.2, specifically, to the nucleotide sequence as set forth in SEQ ID NO:1 or in SEQ ID NO:2, which correspond to the sequences of the above indicated NCBI Reference Sequences of the IL17RE transcript, and also relates to the corresponding amino acid sequence for example as set forth in SEQ ID NO:3 or in SEQ ID NO:4, which correspond to the protein sequences defined in NCBI Protein Accession Reference Sequence NP_705613.1 and in NCBI Protein Accession Reference Sequence NP_001180309.1 encoding the IL17RE polypeptide.

The term “IL17RE” also comprises nucleotide sequences showing a high degree of homology to IL17RE, e.g., nucleic acid sequences being at least 75%, 80%, 85%, 90%, 91%, 92%, 93%, 94%, 95%, 96%, 97%, 98% or 99% identical to the sequence as set forth in SEQ ID NO:1 or in SEQ ID NO:2 or amino acid sequences being at least 75%, 80%, 85%, 90%, 91%, 92%, 93%, 94%, 95%, 96%, 97%, 98% or 99% identical to the sequence as set forth in SEQ ID NO:3 or in SEQ ID NO:4 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:3 or in SEQ ID NO:4 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:1 or in SEQ ID NO:2.

Alternatively, the term “IL117RE” refers to artificial variants of a naturally occurring IL17RE. For example, consensus or core sequences over several isoform sequences can be computationally mapped and defined. Examples of such computationally mapped potential isoform sequences are set forth in SEQ ID NO:5, in SEQ ID NO:6, in SEQ ID NO:7, in SEQ ID NO:8 and in SEQ ID NO:9. A sequence comprising this sequence, or a sequence having at least 90%, 91%, 92%, 93%, 94%, 95%, 96%, 97%, 98% or 99% sequence identity thereto also means an IL17RE in the sense of the present disclosure.

The term “IL1B” refers to the human Interleukin 1 Beta gene (Ensembl: ENSG00000125538), for example, to the sequence as defined in NCBI Reference Sequence NM_000576.3, specifically, to the nucleotide sequence as set forth in SEQ ID NO:10, which corresponds to the sequence of the above indicated NCBI Reference Sequence of the IL1B transcript, and also relates to the corresponding amino acid sequence for example as set forth in SEQ ID NO:11, which corresponds to the protein sequence defined in NCBI Protein Accession Reference Sequence NP_000567.1 encoding the IL1B polypeptide.

The term “IL1B” also comprises nucleotide sequences showing a high degree of homology to IL1B, e.g., nucleic acid sequences being at least 75%, 80%, 85%, 90%, 91%, 92%, 93%, 94%, 95%, 96%, 97%, 98% or 99% identical to the sequence as set forth in SEQ ID NO:10 or amino acid sequences being at least 75%, 80%, 85%, 90%, 91%, 92%, 93%, 94%, 95%, 96%, 97%, 98% or 99% identical to the sequence as set forth in SEQ ID NO:11 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:11 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:10.

Alternatively, the term “IL1B” refers to artificial variants of a naturally occurring IL1B. For example, consensus or core sequences over several isoform sequences can be computationally mapped and defined. Examples of such computationally mapped potential isoform sequences are set forth in SEQ ID NO:12, in SEQ ID NO:13 and in SEQ ID NO:14. A sequence comprising this sequence, or a sequence having at least 90%, 91%, 92%, 93%, 94%, 95%, 96%, 97%, 98% or 99% sequence identity thereto also means an IL1B in the sense of the present disclosure.

The term “IL3” refers to the human Interleukin 3 gene (Ensembl: ENSG00000164399), for example, to the sequence as defined in NCBI Reference Sequence NM_000588.4, specifically, to the nucleotide sequence as set forth in SEQ ID NO:15, which corresponds to the sequence of the above indicated NCBI Reference Sequence of the IL3 transcript, and also relates to the corresponding amino acid sequence for example as set forth in SEQ ID NO:16, which corresponds to the protein sequence defined in NCBI Protein Accession Reference Sequence NP_000579.2 encoding the IL3 polypeptide.

The term “IL3” also comprises nucleotide sequences showing a high degree of homology to IL3, e.g., nucleic acid sequences being at least 75%, 80%, 85%, 90%, 91%, 92%, 93%, 94%, 95%, 96%, 97%, 98% or 99% identical to the sequence as set forth in SEQ ID NO:15 or amino acid sequences being at least 75%, 80%, 85%, 90%, 91%, 92%, 93%, 94%, 95%, 96%, 97%, 98% or 99% identical to the sequence as set forth in SEQ ID NO:16 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:16 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:15.

The term “IL7R” refers to the human Interleukin 7 Receptor gene (Ensembl: ENSG00000168685), for example, to the sequence as defined in NCBI Reference Sequence NM_002185.5, specifically, to the nucleotide sequence as set forth in SEQ ID NO:17, which corresponds to the sequence of the above indicated NCBI Reference Sequence of the IL7R transcript, and also relates to the corresponding amino acid sequence for example as set forth in SEQ ID NO:18, which corresponds to the protein sequence defined in NCBI Protein Accession Reference Sequence NP_002176.2 encoding the IL7R polypeptide.

The term “IL7R” also comprises nucleotide sequences showing a high degree of homology to IL7R, e.g., nucleic acid sequences being at least 75%, 80%, 85%, 90%, 91%, 92%, 93%, 94%, 95%, 96%, 97%, 98% or 99% identical to the sequence as set forth in SEQ ID NO:17 or amino acid sequences being at least 75%, 80%, 85%, 90%, 91%, 92%, 93%, 94%, 95%, 96%, 97%, 98% or 99% identical to the sequence as set forth in SEQ ID NO:18 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:18 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:17.

Alternatively, the term “IL7R” refers to artificial variants of a naturally occurring IL7R. For example, consensus or core sequences over several isoform sequences can be computationally mapped and defined. Examples of such computationally mapped potential isoform sequences are set forth in SEQ ID NO:19, in SEQ ID NO:20, in SEQ ID NO:21, in SEQ ID NO:22, in SEQ ID NO:23 and in SEQ ID NO:24. A sequence comprising this sequence, or a sequence having at least 90%, 91%, 92%, 93%, 94%, 95%, 96%, 97%, 98% or 99% sequence identity thereto also means an IL7R in the sense of the present disclosure.

The term “IL9R” refers to the human Interleukin 9 Receptor gene (ENSG00000124334), for example, to the sequence as defined in NCBI Reference Sequence NM_176786.2 or in NCBI Reference Sequence NM_002186.3, specifically, to the nucleotide sequence as set forth in SEQ ID NO:25 or in SEQ ID NO:26, which correspond to the sequences of the above indicated NCBI Reference Sequences of the IL9R transcript, and also relates to the corresponding amino acid sequence for example as set forth in SEQ ID NO:27 or in SEQ ID NO:28, which correspond to the protein sequences defined in NCBI Protein Accession Reference Sequence NP_789743.2 and in NCBI Protein Accession Reference Sequence NP_002177.2 encoding the IL9R polypeptide.

The term “IL9R” also comprises nucleotide sequences showing a high degree of homology to IL9R, e.g., nucleic acid sequences being at least 75%, 80%, 85%, 90%, 91%, 92%, 93%, 94%, 95%, 96%, 97%, 98% or 99% identical to the sequence as set forth in SEQ ID NO:25 or in SEQ ID NO:26 or amino acid sequences being at least 75%, 80%, 85%, 90%, 91%, 92%, 93%, 94%, 95%, 96%, 97%, 98% or 99% identical to the sequence as set forth in SEQ ID NO:27 or in SEQ ID NO:28 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:27 or in SEQ ID NO:28 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:25 or in SEQ ID NO:26.

The term “EBI3” refers to the human Epstein-Barr Virus Induced 3 gene (Ensembl: ENSG00000105246), for example, to the sequence as defined in NCBI Reference Sequence NM_005755.3, specifically, to the nucleotide sequence as set forth in SEQ ID NO:29, which corresponds to the sequence of the above indicated NCBI Reference Sequence of the EBI3 transcript, and also relates to the corresponding amino acid sequence for example as set forth in SEQ ID NO:30, which corresponds to the protein sequence defined in NCBI Protein Accession Reference Sequence NP_005746.2 encoding the EBI3 polypeptide.

The term “EBI3” also comprises nucleotide sequences showing a high degree of homology to EBI3, e.g., nucleic acid sequences being at least 75%, 80%, 85%, 90%, 91%, 92%, 93%, 94%, 95%, 96%, 97%, 98% or 99% identical to the sequence as set forth in SEQ ID NO:29 or amino acid sequences being at least 75%, 80%, 85%, 90%, 91%, 92%, 93%, 94%, 95%, 96%, 97%, 98% or 99% identical to the sequence as set forth in SEQ ID NO:30 or nucleic acid sequences encoding amino acid sequences being at least 75%, 80%, 85%, 90%, 91%, 92%, 93%, 94%, 95%, 96%, 97%, 98% or 99% identical to the sequence as set forth in SEQ ID NO:30 or amino acid sequences being encoded by nucleic acid sequences being at least 75%, 80%, 85%, 90%, 91%, 92%, 93%, 94%, 95%, 96%, 97%, 98% or 99% identical to the sequence as set forth in SEQ ID NO:29.

The term “biological sample” or “sample obtained from a subject” refers to any biological material obtained via suitable methods known to the person skilled in the art from a subject, e.g., a prostate cancer patient. The biological sample used may be collected in a clinically acceptable manner, e.g., in a way that nucleic acids (in particular RNA) or proteins are preserved.

The biological sample(s) may include body tissue and/or a fluid, such as, but not limited to, blood, sweat, saliva, and urine. Furthermore, the biological sample may contain a cell extract derived from or a cell population including an epithelial cell, such as a cancerous epithelial cell or an epithelial cell derived from tissue suspected to be cancerous. The biological sample may contain a cell population derived from a glandular tissue, e.g., the sample may be derived from the prostate of a male subject. Additionally, cells may be purified from obtained body tissues and fluids if necessary, and then used as the biological sample. In some realizations, the sample may be a tissue sample, a urine sample, a urine sediment sample, a blood sample, a saliva sample, a semen sample, a sample including circulating tumour cells, extracellular vesicles, a sample containing prostate secreted exosomes, or cell lines or cancer cell line.

In one particular realization, biopsy or resections samples may be obtained and/or used. Such samples may include cells or cell lysates.

It is also conceivable that the content of a biological sample is submitted to an enrichment step. For instance, a sample may be contacted with ligands specific for the cell membrane or organelles of certain cell types, e.g., prostate cells, functionalized for example with magnetic particles. The material concentrated by the magnetic particles may subsequently be used for detection and analysis steps as described herein above or below.

Furthermore, cells, e.g., tumour cells, may be enriched via filtration processes of fluid or liquid samples, e.g., blood, urine, etc. Such filtration processes may also be combined with enrichment steps based on ligand specific interactions as described herein above.

The term “prostate cancer” refers to a cancer of the prostate gland in the male reproductive system, which occurs when cells of the prostate mutate and begin to multiply out of control. Typically, prostate cancer is linked to an elevated level of prostate-specific antigen (PSA). In one embodiment of the present invention the term “prostate cancer” relates to a cancer showing PSA levels above 3.0. In another embodiment the term relates to cancer showing PSA levels above 2.0. The term “PSA level” refers to the concentration of PSA in the blood in ng/ml.

The term “non-progressive prostate cancer state” means that a sample of an individual does not show parameter values indicating “biochemical recurrence” and/or “clinical recurrence” and/or “metastases” and/or “castration-resistant disease” and/or “prostate cancer or disease specific death”.

The term “progressive prostate cancer state” means that a sample of an individual shows parameter values indicating “biochemical recurrence” and/or “clinical recurrence” and/or “metastases” and/or “castration-resistant disease” and/or “prostate cancer or disease specific death”.

The term “biochemical recurrence” generally refers to recurrent biological values of increased PSA indicating the presence of prostate cancer cells in a sample. However, it is also possible to use other markers that can be used in the detection of the presence or that rise suspicion of such presence.

The term “clinical recurrence” refers to the presence of clinical signs indicating the presence of tumour cells as measured, for example using in vivo imaging.

The term “metastases” refers to the presence of metastatic disease in organs other than the prostate.

The term “castration-resistant disease” refers to the presence of hormone-insensitive prostate cancer; i.e., a cancer in the prostate that does not any longer respond to androgen deprivation therapy (ADT).

The term “prostate cancer specific death or disease specific death” refers to death of a patient from his prostate cancer.

It is preferred that the determining of the prediction of the radiotherapy response comprises combining the gene expression profiles for three or more, for example, 3, 4, 5 or all, of the interleukin genes with a regression function that had been derived from a population of prostate cancer subjects.

Regression analysis helps one understand how the typical value of the dependent variable (or “criterion variable”) changes when any one of the independent variables is varied, while the other independent variables are held fixed. This relationship between the dependent variable and the independent variables is captured in the regression function, which can be used to predict the dependent variable given the values of the independent variables. The dependent variable can be, for example, a binary variable, such as biochemical relapse within 5 years after radiotherapy. In this case, the regression is a logistic regression that is based on a logit function of the independent variables, which, here, comprise or consist of the gene expression profiles for two or more of the interleukin genes. By means of the regression function, an improved prediction of e.g. the 5-year risk of biochemical recurrence after radiotherapy may be possible.

In one particular realization, the prediction of the radiotherapy response is determined as follows:

c+(w₁·IL17 RE)+(w₂·IL1B)+(w₃·IL3)+(w₄·IL7R)+(w₅·IL9R)+(w₆·EBI3) (1)

where w₁ to w₆ are weights, c is a constant, and IL17RE, IL1B, IL3, IL7R, IL9R, and EBI3 are the expression levels of the interleukin genes.

In one example, w₁ may be about 0.5 to 1.5, such as 9.94141, w₂ may be about −2.0 to −1.0, such as −1.42739, w₃ may be about 1.0 to 2.0, such as 1.26008, w₄ may be about −2.5 to −1.5, such as −1.91264, w₅ may be about 0.0 to 1.0, such as 0.50106, w₆ may be about 4.0 to 6.0, such as 5.10369, and c may be about −6.0 to −4.0, such as −5.174.

The prediction of the radiotherapy response may also be classified or categorized into one of at least two risk groups, based on the value of the prediction of the radiotherapy response. For example, there may be two risk groups, or three risk groups, or four risk groups, or more than four predefined risk groups. Each risk group covers a respective range of (non-overlapping) values of the prediction of the radiotherapy response. For example, a risk group may indicate a probability of occurrence of a specific clinical event from 0 to <0.1 or from 0.1 to <0.25 or from 0.25 to <0.5 or from 0.5 to 1.0 or the like.

It is further preferred that the determining of the prediction of the radiotherapy response is further based on one or more clinical parameters obtained from the subject.

As mentioned above, various measures based on clinical parameters have been investigated. By further basing the prediction of the radiotherapy response on such clinical parameter(s), it can be possible to further improve the prediction.

It is preferred that the one or more clinical parameters comprise one or more of: (i) a prostate-specific antigen (PSA) level; (ii) a pathologic Gleason score (pGS); iii) a clinical tumour stage; iv) a pathological Gleason grade group (pGGG); v) a pathological stage; vi) one or more pathological variables, for example, a status of surgical margins and/or a lymph node invasion and/or an extra-prostatic growth and/or a seminal vesicle invasion; vii) CAPRA-S; and viii) another clinical risk score.

It is further preferred that the determining of the prediction of the radiotherapy response comprises combining the gene expression profiles for the three or more interleukin genes and the one or more clinical parameters obtained from the subject with a regression function that had been derived from a population of prostate cancer subjects.

In one particular realization, the prediction of the radiotherapy response is determined as follows:

c+(w₁·IL17RE)+(w₂·IL1B)+(w₃·IL3)+(w₄·IL7R)+(w₅·IL9R)+(w₆·EBI3)+(w₇·pGGG) (2)

where w₁ to w₇ are weights, c is a constant, IL17RE, IL1B, IL3, IL7R, IL9R and EBI3 are the expression levels of the interleukin genes, and pGGG is the pathological Gleason grade group.

In one example, w₁ may be about 1.5 to 2.5, such as 1.88159, w₂ may be about −0.5 to 0.5, such as −0.041318, w₃ may be about 0.0 to 1.0, such as 0.59493, w₄ may be about −5.0 to −4.0, such as −4.75981, w₅ may be about −1.5 to −0.5, such as −0.84648, w₆ may be about 3.5 to 5.5, such as 4.56796, w₇ may be about 2.0 to 3.0, such as 2.37674, and c may be about −8.5 to −6.5, such as −7.60381.

It is preferred that the biological sample is obtained from the subject before the start of the radiotherapy. The gene expression profiles may be determined in the form of mRNA or protein in tissue of prostate cancer. Alternatively, if the interleukins are present in a soluble form, the gene expression profiles may be determined in blood.

It is further preferred that the radiotherapy is radical radiotherapy or salvage radiotherapy.

It is preferred that the prediction of the radiotherapy response is negative or positive for the effectiveness of the radiotherapy, wherein a therapy is recommended based on the prediction and, if the prediction is negative, the recommended therapy comprises one or more of: (i) radiotherapy provided earlier than is the standard; (ii) radiotherapy with an increased radiation dose; (iii) an adjuvant therapy, such as androgen deprivation therapy; and iv) an alternative therapy that is not a radiation therapy. The degree to which the prediction is negative may determine the degree to which the recommended therapy deviates from the standard form of radiotherapy.

In a further aspect of the present invention, an apparatus for predicting a response of a prostate cancer subject to radiotherapy is presented, comprising:
an input adapted to receive data indicative of a gene expression profile for each of three or more, for example, 3, 4, 5 or all, interleukin genes selected from the group consisting of: IL17RE, IL1B, IL3, IL7R, IL9R, and EBI3, said gene expression profiles being determined in a biological sample obtained from the subject,
a processor adapted to determine the prediction of the radiotherapy response based on the gene expression profiles for the three or more interleukin genes, and
optionally, a providing unit adapted to provide the prediction or a therapy recommendation based on the prediction to a medical caregiver or the subject.

In a further aspect of the present invention, a computer program product is presented comprising instructions which, when the program is executed by a computer, cause the computer to carry out a method comprising:
receiving data indicative of a gene expression profile for each of three or more, for example, 3, 4, 5 or all, interleukin genes selected from the group consisting of: IL17RE, IL1B, IL3, IL7R, IL9R, and EBI3, said gene expression profiles being determined in a biological sample obtained from a prostate cancer subject,
determining the prediction of the radiotherapy response based on the gene expression profiles for the three or more interleukin genes, and
optionally, providing the prediction or a therapy recommendation based on the prediction to a medical caregiver or the subject.

In a further aspect of the present invention, a diagnostic kit is presented, comprising:
at least three primers and/or probes for determining the gene expression profile for each of three or more, for example, 3, 4, 5 or all, interleukin genes selected from the group consisting of: IL17RE, IL1B, IL3, IL7R, IL9R, and EBI3, in a biological sample obtained from the subject, and
an apparatus as defined in claim 10 or a computer program product as defined in claim 11.

In a further aspect of the present invention, a use of the kit as defined in claim 11 is presented.

It is preferred that the use as defined in claim 13 is in a method of predicting a response of a prostate cancer subject to radiotherapy.

In a further aspect of the present invention, a method is presented, comprising:
receiving a biological sample obtained from a prostate cancer subject,
using the kit as defined in claim 12 to determine a gene expression profile for each of three or more, for example, 3, 4, 5 or all, interleukin genes selected from the group consisting of: IL17RE, IL1B, IL3, IL7R, IL9R, and EBI3, in the biological sample obtained from the subject.

In a further aspect of the present invention, a use of a gene expression profile for each of three or more, for example, 3, 4, 5 or all, interleukin genes selected from the group consisting of: IL17RE, IL1B, IL3, IL7R, IL9R, and EBI3, in a method of predicting a response of a prostate cancer subject to radiotherapy is presented, comprising
determining, preferably by a processor, the prediction of the radiotherapy response based on the gene expression profiles for the three or more interleukin genes, and
optionally, providing the prediction or a therapy recommendation based on the prediction to a medical caregiver or the subject.

It shall be understood that the method of claim 1, the apparatus of claim 10, the computer program product of claim 11, the diagnostic kit of claim 12, the use of the diagnostic kit of claim 13, the method of claim 15, and the use of a gene expression profile(s) of claim 16 have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily a flowchart of an embodiment of a method of predicting a response of a prostate cancer subject to radiotherapy.

FIG. 2 shows a ROC curve analysis of three predictive models.

FIG. 3 shows a Kaplan-Meier curve analysis of the Interleukin model (IL_model). The clinical endpoint that was tested was the time to metastases (TTM) after the start of salvage radiation therapy (SRT) due to post-surgical disease recurrence.

FIG. 4 shows a Kaplan-Meier curve analysis of the Interleukin model & pGGG combination model (IL&pGGG_model). The clinical endpoint that was tested was the time to metastases (TTM) after the start of salvage radiation therapy (SRT) due to post-surgical disease recurrence.

FIG. 5 shows a Kaplan-Meier curve analysis of the Interleukin model (IL_model). The clinical endpoint that was tested was the time to prostate cancer specific death (PCa Death) after the start of the salvage radiation therapy (SRT) due to post-surgical disease recurrence.

FIG. 6 shows a Kaplan-Meier curve of the Interleukin model & pGGG combination model (IL&pGGG_model). The clinical endpoint that was tested was the time to prostate cancer specific death (PCa Death) after the start of salvage radiation therapy (SRT) due to post-surgical disease recurrence.

FIG. 7 shows a Kaplan-Meier curve of a Interleukin 3 gene model (IL_3.1_model). The clinical endpoint that was tested was the time to prostate cancer specific death (PCa Death) after the start of salvage radiation therapy (SRT) due to post-surgical disease recurrence.

FIG. 8 shows a Kaplan-Meier curve of another Interleukin 3 gene model (IL_3.2_model). The clinical endpoint that was tested was the time to prostate cancer specific death (PCa Death) after the start of salvage radiation therapy (SRT) due to post-surgical disease recurrence.

FIG. 9 shows a Kaplan-Meier curve of another Interleukin 3 gene model (IL_3.3_model). The clinical endpoint that was tested was the time to prostate cancer specific death (PCa Death) after the start of salvage radiation therapy (SRT) due to post-surgical disease recurrence.

FIG. 10 shows a Kaplan-Meier curve of another DNA Interleukin 3 gene model (IL_3.4_model). The clinical endpoint that was tested was the time to prostate cancer specific death (PCa Death) after the start of salvage radiation therapy (SRT) due to post-surgical disease recurrence.

FIG. 11 shows a Kaplan-Meier curve of another Interleukin 3 gene model (IL_3.5_model). The clinical endpoint that was tested was the time to prostate cancer specific death (PCa Death) after the start of salvage radiation therapy (SRT) due to post-surgical disease recurrence.

FIG. 12 shows a Kaplan-Meier curve of another Interleukin 3 gene model (IL_3.6_model). The clinical endpoint that was tested was the time to prostate cancer specific death (PCa Death) after the start of salvage radiation therapy (SRT) due to post-surgical disease recurrence.

FIG. 13 shows a Kaplan-Meier curve of another Interleukin 3 gene model (IL_3.7_model). The clinical endpoint that was tested was the time to prostate cancer specific death (PCa Death) after the start of salvage radiation therapy (SRT) due to post-surgical disease recurrence.

FIG. 14 shows a Kaplan-Meier curve of another DNA Interleukin 3 gene model (IL_3.8_model). The clinical endpoint that was tested was the time to prostate cancer specific death (PCa Death) after the start of salvage radiation therapy (SRT) due to post-surgical disease recurrence.

### DETAILED DESCRIPTION OF EMBODIMENTS

### Overview of Radiotherapy Response Prediction

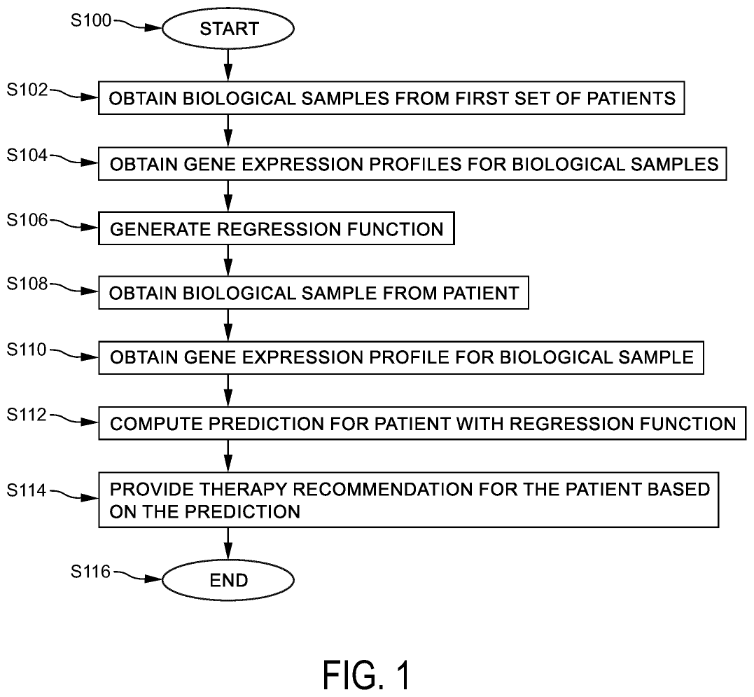

FIG. 1 shows schematically and exemplarily a flowchart of an embodiment of a method of predicting a response of a prostate cancer subject to radiotherapy. The method begins at step S100.

At step S102, a biological sample is obtained from each of a first set of patients (subjects) diagnosed with prostate cancer. Preferably, monitoring prostate cancer has been performed for these prostate cancer patients over a period of time, such as at least one year, or at least two years, or about five years, after obtaining the biological sample.

At step S104, a gene expression profile for each of three or more, for example, 3, 4, 5 or all, interleukin genes selected from the group consisting of: IL17RE, IL1B, IL3, IL7R, IL9R, and EBI3, is obtained for each of the biological samples obtained from the first set of patients, e.g., by performing RT-qPCR (real-time quantitative PCR) on RNA extracted from each biological sample. The exemplary gene expression profiles include an expression level (e.g., value) for each of the two or more interleukin genes.

At step S106, a regression function for assigning a prediction of the radiotherapy response is determined based on the gene expression profiles for the three or more interleukin genes, IL17RE, IL1B, IL3, IL7R, IL9R, and/or EBI3, obtained for at least some of the biological samples obtained for the first set of patients and respective results obtained from the monitoring. In one particular realization, the regression function is determined as specified in Eq. (1) above.

At step S108, a biological sample is obtained from a patient (subject or individual). The patient can be a new patient or one of the first set.

At step S110, a gene expression profile is obtained for each of the three or more, for example, 3, 4, 5 or all, interleukin genes, e.g., by performing PCR on the biological sample.

At step S112, a prediction of the radiotherapy response based on the gene expression profiles for the two or more interleukin genes is determined for the patient using the regression function. This will be described in more detail later in the description.

At S114, a therapy recommendation may be provided, e.g., to the patient or his or her guardian, to a doctor, or to another healthcare worker, based on the prediction. To this end, the prediction may be categorized into one of a predefined set of risk groups, based on the value of the prediction. In one particular realization, the prediction of the radiotherapy response may be negative or positive for the effectiveness of the radiotherapy. If the prediction is negative, the recommended therapy may comprise one or more of: (i) radiotherapy provided earlier than is the standard; (ii) radiotherapy with an increased radiation dose; (iii) an adjuvant therapy, such as androgen deprivation therapy; and iv) an alternative therapy that is not a radiation therapy.

The method ends at S116.

In one embodiment, the gene expression profiles at steps S104 and S110 are determined by detecting mRNA expression using eight or more primers and/or probes and/or eight or more sets thereof.

In one embodiment, steps S104 and S110 further comprise obtaining one or more clinical parameters from the first set of patients and the patient, respectively. The one or more clinical parameters may comprise one or more of: (i) a prostate-specific antigen (PSA) level; (ii) a pathologic Gleason score (pGS); iii) a clinical tumour stage; iv) a pathological Gleason grade group (pGGG); v) a pathological stage; vi) one or more pathological variables, for example, a status of surgical margins and/or a lymph node invasion and/or an extra-prostatic growth and/or a seminal vesicle invasion; vii) CAPRA-S; and viii) another clinical risk score. The regression function for assigning the prediction of the radiotherapy response that is determined in step S106 is then further based on the one or more clinical parameters obtained from at least some of the first set of patients. In step S112, the prediction of the radiotherapy response is then further based on the one or more clinical parameters, e.g., the pathological Gleason grade group (pGGG), obtained from the patient and is determined for the patient using the regression function.

The immune system interacts in a strong manner with prostate cancer, both on a systemic level and in the tumour microenvironment. Interleukins play a central role in the regulation of immune activity. Interleukins and their receptors may therefore provide information on the effectiveness of RT. However, which members of the interleukin family may have predictive value in this application is extremely difficult to deduce from existing literature due to the many factors that influence the exact function of interleukins.

We investigated the extent to which the expression of interleukins and their receptors in prostate cancer tissue correlates with the recurrence of disease after radical RT or SRT.

We have identified six members of the family of interleukins and interleukin receptors for which the degree of expression in prostate cancer tissue significantly correlates with mortality after SRT, in a cohort of 151 prostate cancer patients. For two of these interleukins, we found also a significant correlation of expression with disease recurrence in an independent cohort of 248 patients treated with radical RT for primary localized prostate cancer. One of these two interleukin receptors showed in addition a significant correlation with the occurrence of metastases after SRT in the first cohort of 151 patients.

Based on the significant correlation with outcome after RT, we expect that the identified molecules will provide predictive value with regard to the effectiveness of radical RT and/or SRT.

TABLE 1
Univariate Cox regression analysis of two independent prostate cancer patient cohorts.
The number of patients are indicated per cohort. The tested endpoint in the two
cohorts is post-treatment progression free survival for men undergoing salvage
radiation (SRT) after post-surgical disease recurrence (cohort #1) vs. men
stratified to radical radiation (RRT) as the primary, localized therapy (cohort
#2). The number of events and percentage per endpoint is indicated in parenthesis.
The table indicates for each tested interleukin gene the association in terms of
risk (Hazard Ratio) and significance (p-value) to the tested endpoints.

| | Data Set | | | | | | | |
|---|---|---|---|---|---|---|---|---|
| | #1 (Prostate RNAseq) | | | | #2 (GSE116918) | | | |
| | # Patients | | | | | | | |
| | 151 | | | | 248 | | | |
| | Outcome | | | | | | | |
| | Post-Salvage-Radiation Outcome | | | | Post-Primary-Radiation Outcome | | | |
| | Endpoint (# events/# patients; % events) | | | | | | | |
| | Metastases | | Prostate Cancer | | Biochemical | | Metastases | |
| | (#65/#151; | | Mortality | | Relapse (#56/#248; | | (#22/#248; | |
| | 43.0%) | | (#26/#151; 17.2%) | | 22.6%) | | 8.9%) | |
| Interleukin | p-value | HR | p-value | HR | p-value | HR | p-value | HR |
| IL17RE | 0.003 | 1.84 | 0.0009 | 2.26 | 0.029 | 2.26 | 0.049 | 3.14 |
| IL1B | 0.740 | 1.07 | 0.0081 | 0.21 | 0.533 | 1.08 | 0.070 | 1.31 |
| IL3 | 0.100 | 1.35 | 0.018 | 1.56 | 0.843 | 0.92 | 0.832 | 1.15 |
| IL7R | 0.550 | 0.78 | 0.023 | 0.12 | 0.256 | 1.15 | 0.350 | 1.20 |
| IL9R | 0.100 | 1.42 | 0.025 | 1.71 | 0.086 | 1.55 | 0.043 | 2.03 |
| EBI3 | 0.250 | 2.49 | 0.019 | 6.76 | 0.020 | 2.30 | 0.119 | 2.42 |

Compared to Hall and colleagues (see Hall W. A. et al., 2018, ibid), the number of interleukins we tested was larger, encompassing all known interleukins and their receptors. For example, the molecules IL17RE, EBI3, IL7R, IL9R and IL3, which we found to have predictive value, were not analysed by Hall W. A. et al., 2018. Another difference is that they analysed proteins in blood serum, while we analysed mRNA in tissue. We found a predictive value for IL1B but not for IL10, contrary to the findings of Hall W. A. et al., 2018.

### Results

### Logistic Regression Analysis

We then set out to test whether the combination of these six interleukins and interleukin receptors will exhibit more prognostic value. With logistic regression we modelled the expression levels of the six interleukins to 10-year prostate cancer specific death after post-surgical salvage RT either with (IL&pGGG_model) or without (IL_model) the presence of the variable pathological Gleason grade group (pGGG). We tested the two models in ROC curve analysis as well as in Kaplan-Meier survival analysis.

The logit(p) regression functions were derived as follows:

### IL_model:

c+(w₁·IL17RE)+(w₂·IL1B)+(w₃·IL3)+(w₄·IL7R)+(w₅·IL9R)+(w₆·EBI3)

### IL&pGGG_model:

c+(w₁·IL17RE)+(w₂·IL1B)+(w₃·IL3)+(w₄·IL7R)+(w₅·IL9R)+(w₆·EBI3)+(w₇·pGGG)

The details for the weights w₁ to w₇ and the constant c are shown in the following TABLE 2.

TABLE 2
Variables and weights for the two logistic regression models,
i.e., the Interleukin model (IL_model) and the Interleukin & pGGG
combination model (IL&pGGG_model); NA—not available.

| | |
|---|---|
| Variable | Weights |

| Model | | IL_model | IL&pGGG_model |
|---|---|---|---|
| IL17RE | w₁ | 0.94141 | 1.88159 |
| IL1B | w₂ | −1.42739 | −0.041318 |
| IL3 | w₃ | 1.26008 | 0.59493 |
| IL7R | w₄ | −1.91264 | −4.75981 |
| IL9R | w₅ | 0.50106 | −0.84648 |
| EBI3 | w₆ | 5.10369 | 4.56796 |
| pGGG | w₇ | NA | 2.37674 |
| Constant | c | −5.174 | −7.60381 |

### ROC Curve Analysis

Next, we tested the logistic regression models as outlined above for their power to predict 10-year prostate cancer specific death after start of salvage radiation due to post-surgical disease recurrence. The performance of the models was compared to the clinical risk score CAPRA-S (see Cooperberg M. R. et al., “The CAPRA-S score: A straightforward tool for improved prediction of outcomes after radical prostatectomy”, Cancer, Vol. 117, No. 22, pages 5039-5046, 2011).

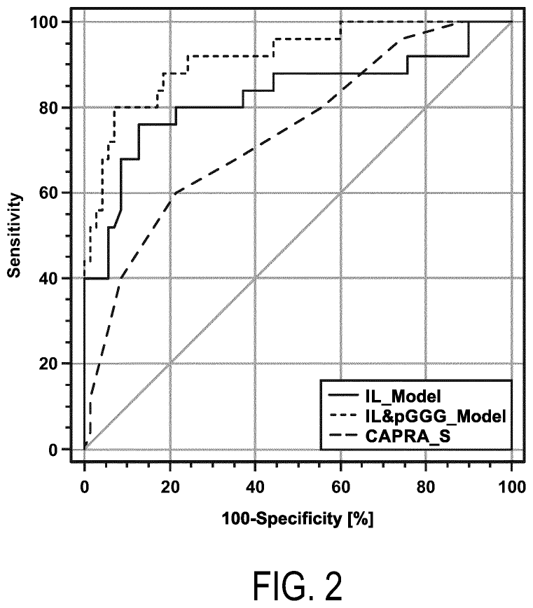

FIG. 2 shows a ROC curve analysis of three predictive models. The IL_model (AUC=0.83) is the logistic regression model based on six interleukins. The IL&pGGG_model (AUC=0.92) is logistic regression model based on six interleukins and the pathology Gleason grade group (pGGG) information. The CAPRA_S (AUC=0.74) is the clinical CAPRA-S score (Cancer of the Prostate Risk Assessment score).

### Kaplan-Meier Survival Analysis

For Kaplan-Meier curve analysis, the logit(p) function of the two risk models (IL_model and IL&pGGG_model) was transferred into risk probabilities and the patient cohort was categorized into four sub-cohorts based on different arbitrarily selected cut-offs (see description of figures below). The goal was to create patient classes with a to some extent similar number of patients within the individual group. For better comparability, the same cut-offs were selected for both models.

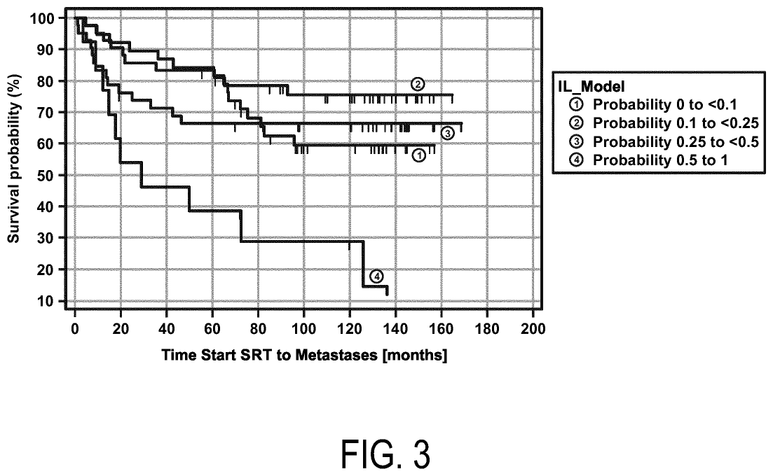

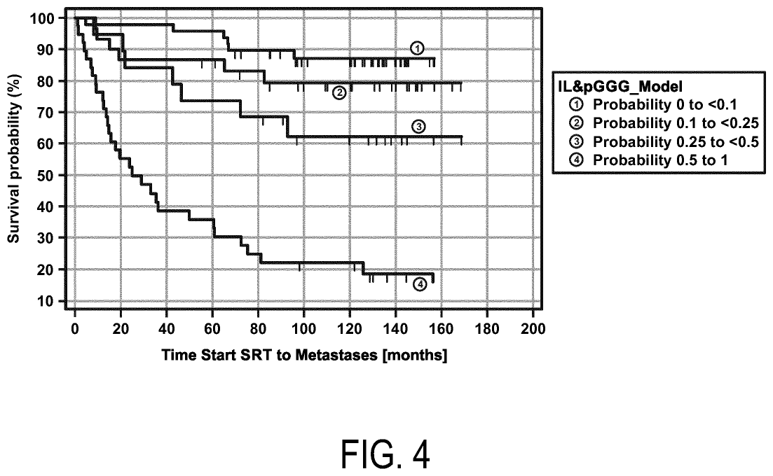

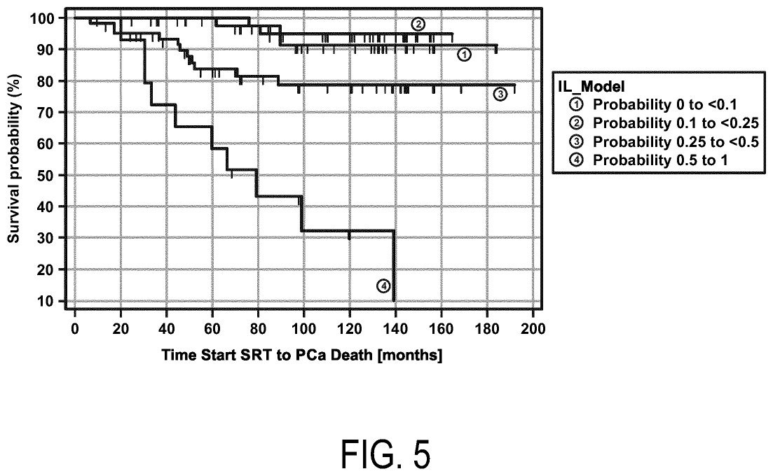

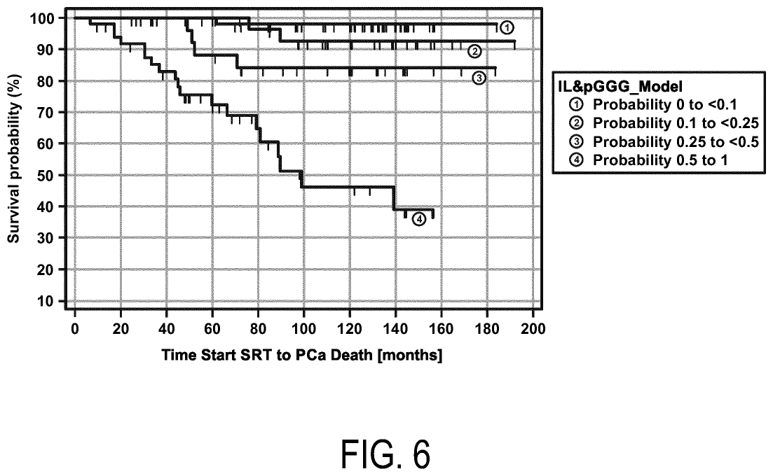

The patient classes represent an increasing risk to experience the tested clinical endpoints of time to development of metastases (FIGS. 3 and 4) or time to prostate cancer specific death (FIGS. 5 and 6) since the start of salvage RT for the two created risk models (IL_model; IL&pGGG_model).

FIG. 3 shows a Kaplan-Meier curve analysis of the IL_model. The clinical endpoint that was tested was the time to metastases (TTM) after the start of salvage radiation therapy (SRT) due to post-surgical disease recurrence. Patients were stratified into four groups according to their risk to experience the clinical endpoint as predicted by the respective logistic regression model. The following supplementary lists indicate the number of patients at risk for the IL_model classes analyzed, i.e., the patients at risk at any time interval +20 months after surgery are shown: Probability 0 to <0.1: 38, 35, 33, 32, 24, 15, 14, 4, 0, 0; Probability 0.1 to <0.25: 42, 38, 35, 34, 31, 26, 24, 11, 1, 0; Probability 0.25 to <0.5: 42, 31, 29, 27, 26, 23, 23, 14, 2, 0; Probability 0.5 to <1: 13, 7, 6, 5, 3, 3, 2, 0, 0, 0.

FIG. 4 shows a Kaplan-Meier curve analysis of the IL&pGGG_model. The clinical endpoint that was tested was the time to metastases (TTM) after the start of salvage radiation therapy (SRT) due to post-surgical disease recurrence. Patients were stratified into four groups according to their risk to experience the clinical endpoint as predicted by the respective logistic regression model. The following supplementary lists indicate the number of patients at risk for the IL&pGGG_model classes analyzed, i.e., the patients at risk at any time interval +20 months after surgery are shown: Probability 0 to <0.1: 48, 47, 47, 46, 40, 33, 32, 14, 0, 0; Probability 0.1 to <0.25: 30, 26, 26, 25, 22, 18, 16, 9, 2, 0; Probability 0.25 to <0.5: 19, 18, 16, 14, 13, 9, 8, 4, 1, 9; Probability 0.5 to <1: 38, 20, 14, 13, 9, 7, 7, 2, 0, 0.

FIG. 5 shows a Kaplan-Meier curve analysis of the IL_model. The clinical endpoint that was tested was the time to prostate cancer specific death (PCa Death) after the start of the salvage radiation therapy (SRT) due to post-surgical disease recurrence. Patients were stratified into four groups according to their risk of experience the clinical endpoint as predicted by the respective logistic regression model. The following supplementary lists indicate the number of patients at risk for the IL_model classes analyzed, i.e., the patients at risk at any time interval +20 months after surgery are shown: Probability 0 to <0.1: 39, 39, 37, 36, 31, 22, 19, 9, 2, 2, 0; Probability 0.1 to <0.25: 43, 42, 39, 36, 34, 30, 25, 13, 1, 0, 0; Probability 0.25 to <0.5: 56, 52, 45, 35, 27, 23, 22, 13, 3, 1, 0; Probability 0.5 to <1: 13, 12, 9, 7, 4, 2, 1, 0, 0, 0, 0.

FIG. 6 shows a Kaplan-Meier curve of the IL&pGGG_model. The clinical endpoint that was tested was the time to prostate cancer specific death (PCa Death) after the start of salvage radiation therapy (SRT) due to post-surgical disease recurrence. Patients were stratified into four groups according to their risk to experience the clinical endpoint as predicted by the respective logistic regression model. The following supplementary lists indicate the number of patients at risk for the IL&pGGG_model classes analyzed, i.e., the patients at risk at any time interval +20 months after surgery are shown: Probability 0 to <0.1: 48, 48, 47, 47, 43, 37, 34, 17, 1, 1, 0; Probability 0.1 to <0.25: 32, 32, 30, 28, 24, 20, 15, 8, 3, 1, 0; Probability 0.25 to <0.5: 26, 26, 23, 20, 16, 13, 11, 6, 2, 1, 0; Probability 0.5 to <1: 45, 39, 30, 19, 13, 7, 7, 4, 0, 0, 0.

The Kaplan-Meier curve analysis as shown in FIGS. 3 to 6 demonstrates the presence of different patient risk groups. The risk group of a patient is determined by the probability to suffer from the respective clinical endpoint (metastases, prostate cancer specific death) as calculated by the risk model IL_model or IL&pGGG_model. Depending on the predicted risk of a patient (i.e., depending on in which risk group 1 to 4 the patient may belong) different types of interventions might be indicated. In the lowest risk groups (probability <0.25) standard of care (SOC), which is SRT potentially combined with SADT (salvage androgen deprivation therapy), delivers acceptable long-term oncological control. For the patient group with a risk between 0.25 and 0.5 dose escalation of the applied RT and/or combination with chemotherapy might provide improved longitudinal outcomes. This is definitely not the case for the patient group with a risk >0.5 to experience any of the relevant outcomes. In this patient group escalation of intervention is indicated. Options for escalation are early combination of SRT (considering higher dose regimens), SADT, and chemotherapy. Other options are alternative therapies like immunotherapies (e.g., Sipuleucil-T) or other experimental therapies.

### Further Results

This section shows additional results for Cox regression models based on only three of the identified interleukin genes, respectively. In total, eight different 3 gene models were tested. The details for the weights are shown in the following TABLE 3.

TABLE 3
Variables and weights for the 3 gene Cox regression models, i.e., the eight
interleukin 3 gene models (IL_3.1_model to IL_3.8_model); NA—not available.

| | IL 3 gene regression models | | | | | | | |
|---|---|---|---|---|---|---|---|---|
| Variable | 3.1 | 3.2 | 3.3 | 3.4 | 3.5 | 3.6 | 3.7 | 3.8 |
| EBI3 | −0.222 | NA | NA | NA | −0.3273 | NA | −0.3036 | −0.4282 |
| IL17RE | −0.2551 | −0.2726 | NA | NA | NA | −0.2777 | −0.3235 | NA |
| IL1B | 0.4862 | 0.4784 | 0.4069 | NA | 0.3648 | NA | NA | NA |
| IL3 | NA | 0.00806 | −0.1907 | −1.0628 | NA | −0.8431 | NA | NA |
| IL7R | NA | NA | 0.1147 | 0.2348 | 0.1939 | NA | NA | 0.3203 |
| IL9R | NA | NA | NA | 1.0979 | NA | 1.0918 | 0.471 | 0.3441 |

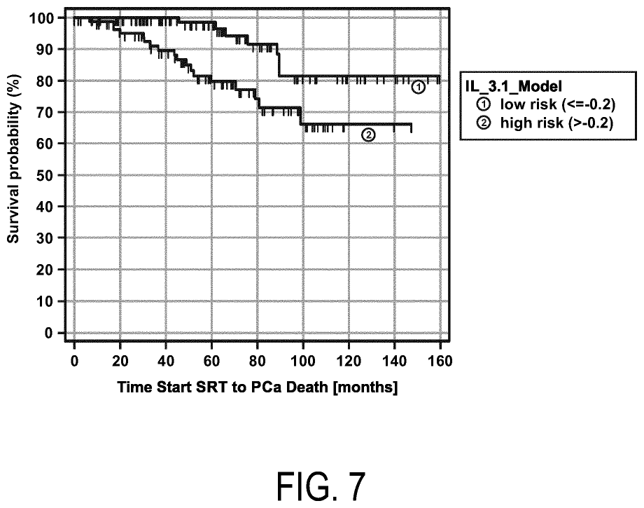

FIG. 7 shows a Kaplan-Meier curve of the IL_3.1_model. The clinical endpoint that was tested was the time to prostate cancer specific death (PCa Death) after the start of salvage radiation therapy (SRT) due to post-surgical disease recurrence. Patients were stratified into two cohorts (low vs. high) according to their risk to experience the clinical endpoint as predicted by the IL_3.1_model using the value −0.2 as cut-off (logrank p=0.008; HR=2.9; CI=1.3-6.4). The following supplementary list indicate the number of patients at risk for the IL_3.1_model classes analyzed, i.e., the patients at risk at any time interval +20 months after surgery are shown: Low risk: 97, 87, 68, 55, 34, 20, 13, 7, 0; High risk: 88, 76, 63, 43, 26, 13, 2, 1, 0.

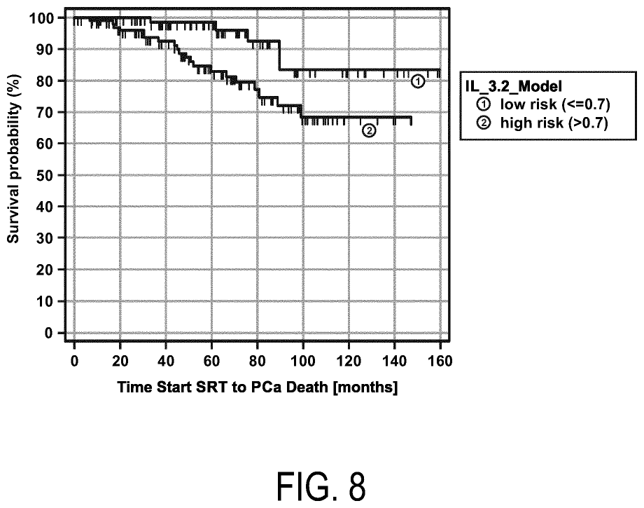

FIG. 8 shows a Kaplan-Meier curve of the IL_3.2_model. The clinical endpoint that was tested was the time to prostate cancer specific death (PCa Death) after the start of salvage radiation therapy (SRT) due to post-surgical disease recurrence. Patients were stratified into two cohorts (low vs. high) according to their risk to experience the clinical endpoint as predicted by the IL_3.2_model using the value 0.7 as cut-off (logrank p=0.02; HR=2.6; CI=1.2-5.7). The following supplementary list indicate the number of patients at risk for the IL_3.2_model classes analyzed, i.e., the patients at risk at any time interval +20 months after surgery are shown: Low risk: 78, 71, 56, 44, 26, 15, 10, 6, 0; High risk: 107, 92, 75, 54, 34, 18, 5, 2, 0.

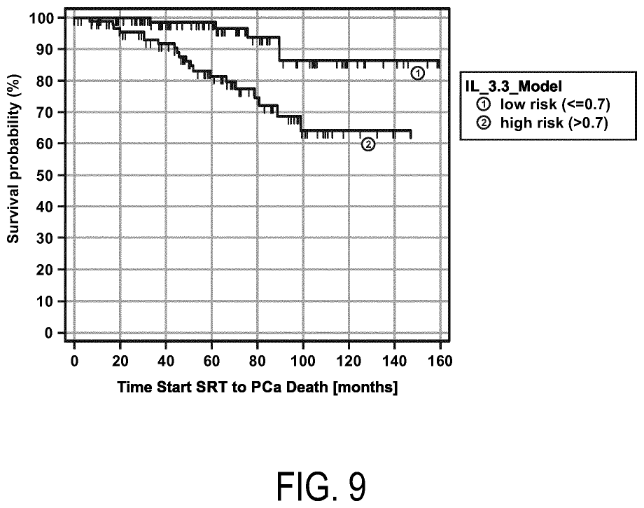

FIG. 9 shows a Kaplan-Meier curve of the IL_3.3_model. The clinical endpoint that was tested was the time to prostate cancer specific death (PCa Death) after the start of salvage radiation therapy (SRT) due to post-surgical disease recurrence. Patients were stratified into two cohorts (low vs. high) according to their risk to experience the clinical endpoint as predicted by the IL_3.3_model using the value 0.7 as cut-off (logrank p=0.002; HR=3.4; CI=1.6-7.5). The following supplementary list indicate the number of patients at risk for the IL_3.3_model classes analyzed, i.e., the patients at risk at any time interval +20 months after surgery are shown: Low risk: 90, 82, 62, 50, 32, 20, 10, 6, 0; High risk: 95, 81, 69, 48, 28, 13, 5, 2, 0.

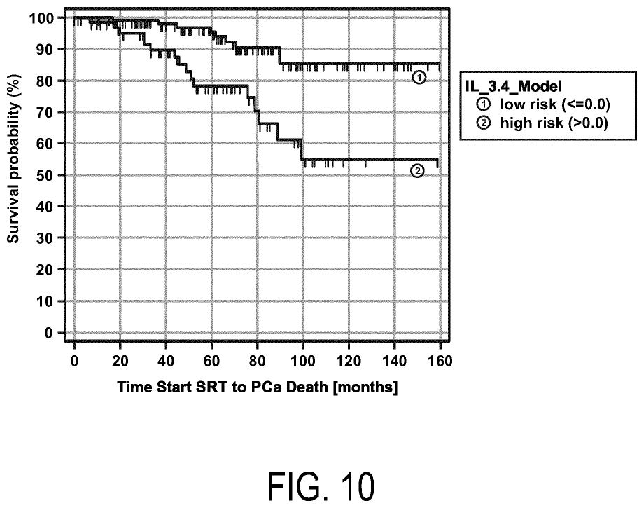

FIG. 10 shows a Kaplan-Meier curve of the IL_3.4_model. The clinical endpoint that was tested was the time to prostate cancer specific death (PCa Death) after the start of salvage radiation therapy (SRT) due to post-surgical disease recurrence. Patients were stratified into two cohorts (low vs. high) according to their risk to experience the clinical endpoint as predicted by the IL_3.4_model using the value 0.0 as cut-off (logrank p=0.002; HR=4.5; CI=1.6-7.6). The following supplementary list indicate the number of patients at risk for the IL_3.4_model classes analyzed, i.e., the patients at risk at any time interval +20 months after surgery are shown: Low risk: 117, 107, 87, 68, 43, 24, 13, 7, 0; High risk: 68, 56, 44, 30, 17, 9, 2, 1, 0.

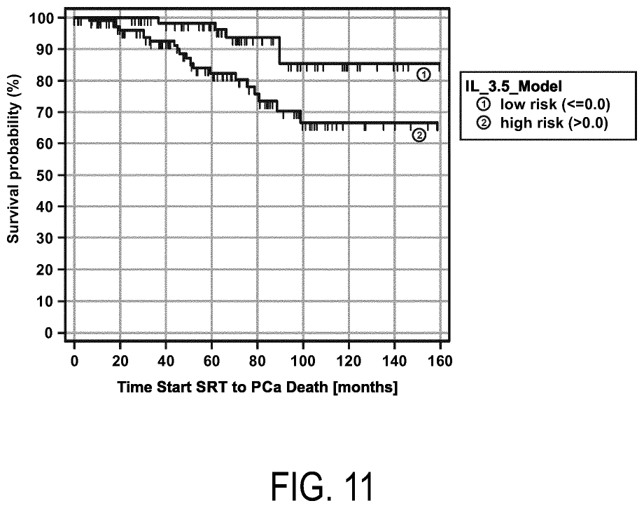

FIG. 11 shows a Kaplan-Meier curve of the IL_3.5_model. The clinical endpoint that was tested was the time to prostate cancer specific death (PCa Death) after the start of salvage radiation therapy (SRT) due to post-surgical disease recurrence. Patients were stratified into two cohorts (low vs. high) according to their risk to experience the clinical endpoint as predicted by the IL_3.5_model using the value 0.0 as cut-off (logrank p=0.0004; HR=4.2; CI=1.9-9.1). The following supplementary list indicate the number of patients at risk for the IL_3.5_model classes analyzed, i.e., the patients at risk at any time interval +20 months after surgery are shown: Low risk: 75, 70, 59, 48, 28, 16, 8, 4, 0; High risk: 110, 93, 72, 50, 32, 17, 7, 4, 0.

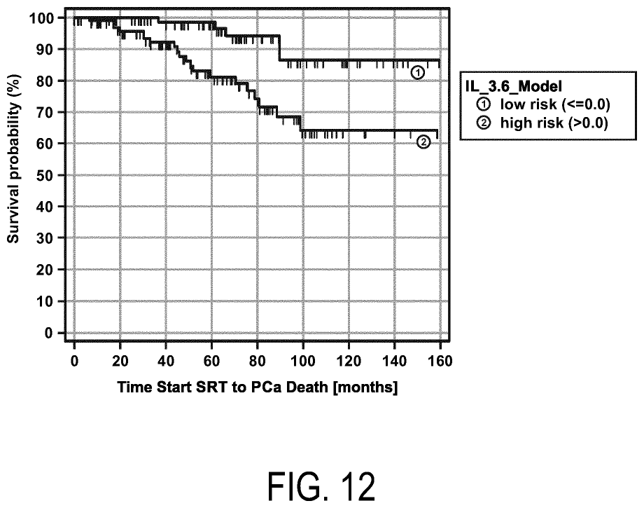

FIG. 12 shows a Kaplan-Meier curve of the IL_3.6_model. The clinical endpoint that was tested was the time to prostate cancer specific death (PCa Death) after the start of salvage radiation therapy (SRT) due to post-surgical disease recurrence. Patients were stratified into two cohorts (low vs. high) according to their risk to experience the clinical endpoint as predicted by the IL_3.6_model using the value 0.0 as cut-off (logrank p=0.03; HR=2.5; CI=1.1-5.5). The following supplementary list indicate the number of patients at risk for the IL_3.6_model classes analyzed, i.e., the patients at risk at any time interval +20 months after surgery are shown: Low risk: 81, 75, 64, 52, 30, 18, 10, 5, 0; High risk: 104, 88, 67, 46, 30, 15, 5, 3, 0.

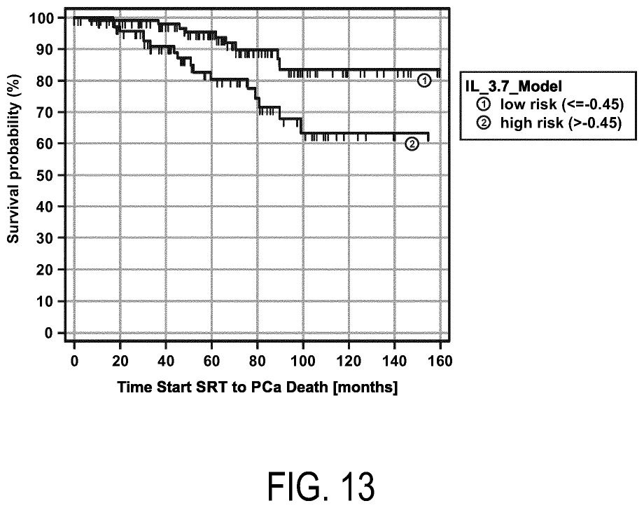

FIG. 13 shows a Kaplan-Meier curve of the IL_3.7_model. The clinical endpoint that was tested was the time to prostate cancer specific death (PCa Death) after the start of salvage radiation therapy (SRT) due to post-surgical disease recurrence. Patients were stratified into two cohorts (low vs. high) according to their risk to experience the clinical endpoint as predicted by the IL_3.7_model using the value −0.45 as cut-off (logrank p=0.01; HR=2.8; CI=1.3-6.3). The following supplementary list indicate the number of patients at risk for the IL_3.7_model classes analyzed, i.e., the patients at risk at any time interval +20 months after surgery are shown: Low risk: 104, 96, 81, 63, 35, 19, 10, 6, 0; High risk: 81, 67, 50, 35, 25, 14, 5, 2, 0.

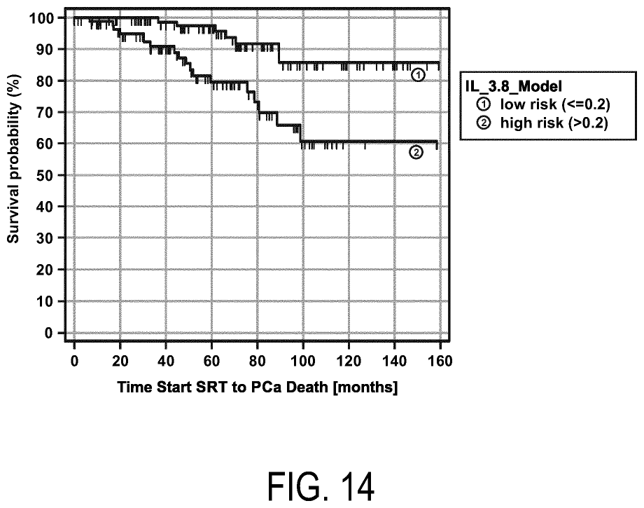

FIG. 14 shows a Kaplan-Meier curve of the IL_3.8_model. The clinical endpoint that was tested was the time to prostate cancer specific death (PCa Death) after the start of salvage radiation therapy (SRT) due to post-surgical disease recurrence. Patients were stratified into two cohorts (low vs. high) according to their risk to experience the clinical endpoint as predicted by the IL_3.8_model using the value 0.2 as cut-off (logrank p<0.001; HR=3.7; CI=1.7-8.2). The following supplementary list indicate the number of patients at risk for the IL_3.8_model classes analyzed, i.e., the patients at risk at any time interval +20 months after surgery are shown: Low risk: 96, 90, 75, 61, 37, 22, 13, 7, 0; High risk: 89, 73, 56, 37, 23, 11, 2, 1, 0.

The Kaplan-Meier analysis as shown in FIGS. 7 to 14 demonstrates that different patient risk groups can also be distinguished using risk models that are only based on a subset of the identified interleukin genes, for example, three of the genes.

### Discussion

The effectiveness of both radical RT and SRT for localized prostate cancer is limited, resulting in disease progression and ultimately death of patients, especially for those at high risk of recurrence. The prediction of the therapy outcome is very complicated as many factors play a role in therapy effectiveness and disease recurrence. It is likely that important factors have not yet been identified, while the effect of others cannot be determined precisely. Multiple clinico-pathological measures are currently investigated and applied in a clinical setting to improve response prediction and therapy selection, providing some degree of improvement. Nevertheless, a strong need remains for better prediction of the response to radical RT and to SRT, in order to increase the success rate of these therapies.

We have identified molecules of which expression shows a significant relation to mortality after radical RT and SRT and therefore are expected to improve the prediction of the effectiveness of these treatments. An improved prediction of effectiveness of RT for each patient be it in the radical or the salvage setting, will improve therapy selection and potentially survival. This can be achieved by 1) optimizing RT for those patients where RT is predicted to be effective (e.g. by dose escalation or a different starting time) and 2) guiding patients where RT is predicted not to be effective to an alternative, potentially more effective form of treatment. Further, this would reduce suffering for those patients who would be spared ineffective therapy and would reduce cost spent on ineffective therapies.

Other variations to the disclosed realizations can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

One or more steps of the method illustrated in FIG. 1 may be implemented in a computer program product that may be executed on a computer. The computer program product may comprise a non-transitory computer-readable recording medium on which a control program is recorded (stored), such as a disk, hard drive, or the like. Common forms of non-transitory computer-readable media include, for example, floppy disks, flexible disks, hard disks, magnetic tape, or any other magnetic storage medium, CD-ROM, DVD, or any other optical medium, a RAM, a PROM, an EPROM, a FLASH-EPROM, or other memory chip or cartridge, or any other non-transitory medium from which a computer can read and use.

Alternatively, the one or more steps of the method may be implemented in transitory media, such as a transmittable carrier wave in which the control program is embodied as a data signal using transmission media, such as acoustic or light waves, such as those generated during radio wave and infrared data communications, and the like.

The exemplary method may be implemented on one or more general purpose computers, special purpose computer(s), a programmed microprocessor or microcontroller and peripheral integrated circuit elements, an ASIC or other integrated circuit, a digital signal processor, a hardwired electronic or logic circuit such as a discrete element circuit, a programmable logic device such as a PLD, PLA, FPGA, Graphical card CPU (GPU), or PAL, or the like. In general, any device, capable of implementing a finite state machine that is in turn capable of implementing the flowchart shown in FIG. 1, can be used to implement one or more steps of the method of risk stratification for therapy selection in a patient with prostate cancer is illustrated. As will be appreciated, while the steps of the method may all be computer implemented, in some embodiments one or more of the steps may be at least partially performed manually.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified herein.

Any reference signs in the claims should not be construed as limiting the scope.

The invention relates to a method of predicting a response of a prostate cancer subject to radiotherapy, comprising determining or receiving the result of a determination of a gene expression profile for each of three or more, for example, 3, 4, 5 or all, interleukin genes selected from the group consisting of: IL17RE, IL1B, IL3, IL7R, IL9R, and EBI3, said gene expression profiles being determined in a biological sample obtained from the subject, determining, preferably by a processor, the prediction of the radiotherapy response based on the gene expression profiles for the three or more interleukin genes, and optionally, providing the prediction or a therapy recommendation based on the prediction to a medical caregiver or the subject. Since the status of the immune system and of the immune microenvironment have an impact on therapy effectiveness, the ability to identify markers predictive for this effect might help to be better able to predict overall RT response. Interleukins play a central role in the regulation of immune activity. The identified interleukins were found to exhibit a significant correlation with outcome after RT, wherefore we expect that they will provide predictive value with regard to the effectiveness of radical RT and/or SRT.

The attached Sequence Listing, entitled 2019PF00710_Sequence Listing_ST25 is incorporated herein by reference, in its entirety.

1. A method of predicting a response of a prostate cancer subject to radiotherapy, comprising:
determining a gene expression profile for each of three or more, for example, 3, 4, 5 or all, interleukin genes selected from the group consisting of: IL17RE, IL1B, IL3, IL7R, IL9R, and EBI3, said gene expression profiles being determined in a biological sample obtained from the subject,
determining, the prediction of the radiotherapy response based on the gene expression profiles for the three or more interleukin genes, and
optionally, providing the prediction or a therapy recommendation based on the prediction to a medical caregiver or the subject.

2. A method of predicting a response of a prostate cancer subject to radiotherapy, comprising:
receiving the result of a determination of a gene expression profile for each of three or more, for example, 3, 4, 5 or all, interleukin genes selected from the group consisting of: IL17RE, IL1B, IL3, IL7R, IL9R, and EBI3, said gene expression profiles being determined in a biological sample obtained from the subject,
determining, by a processor, the prediction of the radiotherapy response based on the gene expression profiles for the three or more interleukin genes, and
optionally, providing the prediction or a therapy recommendation based on the prediction to a medical caregiver or the subject.

3. (canceled)

4. The method as defined in claim 1, wherein the three or more interleukin genes comprise all of the interleukin genes.

5. The method as defined in claim 1, wherein the determining of the prediction of the radiotherapy response comprises combining the gene expression profiles for three or more, for example, 3, 4, 5 or all, of the interleukin genes with a regression function that had been derived from a population of prostate cancer subjects.

6. The method as defined in claim 1, wherein the determining of the prediction of the radiotherapy response is further based on one or more clinical parameters obtained from the subject.

7. The method as defined in claim 6, wherein the one or more clinical parameters comprise one or more of: (i) a prostate-specific antigen (PSA) level; (ii) a pathologic Gleason score (pGS); iii) a clinical tumour stage; iv) a pathological Gleason grade group (pGGG); v) a pathological stage; vi) one or more pathological variables, for example, a status of surgical margins and/or a lymph node invasion and/or an extra-prostatic growth and/or a seminal vesicle invasion; vii) CAPRA-S; and viii) another clinical risk score.

8. The method as defined in claim 6, wherein the determining of the prediction of the radiotherapy response comprises combining the gene expression profiles for the three or more interleukin genes and the one or more clinical parameters obtained from the subject with a regression function that had been derived from a population of prostate cancer subjects.

9. The method as defined in claim 1, wherein the biological sample is obtained from the subject before the start of the radiotherapy.

10. The method as defined in claim 1, wherein the radiotherapy is radical radiotherapy or salvage radiotherapy.

11. The method as defined in claim 1, wherein the prediction of the radiotherapy response is negative or positive for the effectiveness of the radiotherapy, wherein a therapy is recommended based on the prediction and, if the prediction is negative, the recommended therapy comprises one or more of: (i) radiotherapy provided earlier than is the standard; (ii) radiotherapy with an increased radiation dose; (iii) an adjuvant therapy, such as androgen deprivation therapy; and iv) an alternative therapy that is not a radiation therapy.

12. An apparatus for predicting a response of a prostate cancer subject to radiotherapy, comprising:
an input adapted to receive data indicative of a gene expression profile for each of three or more, for example, 3, 4, 5 or all, interleukin genes selected from the group consisting of: IL17RE, IL1B, IL3, IL7R, IL9R, and EBI3, said gene expression profiles being determined in a biological sample obtained from the subject,
a processor adapted to determine the prediction of the radiotherapy response based on the gene expression profiles for the three or more interleukin genes, and
optionally, a providing unit adapted to provide the prediction or a therapy recommendation based on the prediction to a medical caregiver or the subject.

13. A non-transitory computer program product comprising instructions which, when the program is executed by a computer, cause the computer to carry out a method comprising:
receiving data indicative of a gene expression profile for each of three or more, for example, 3, 4, 5 or all, interleukin genes selected from the group consisting of: IL17RE, IL1B, IL3, IL7R, IL9R, and EBI3, said gene expression profiles being determined in a biological sample obtained from a prostate cancer subject,
determining the prediction of the radiotherapy response based on the gene expression profiles for the three or more interleukin genes, and
optionally, providing the prediction or a therapy recommendation based on the prediction to a medical caregiver or the subject.

14. A diagnostic kit, comprising:
at least three primers and/or probes for determining the gene expression profile for each of three or more, for example, 3, 4, 5 or all, interleukin genes selected from the group consisting of: IL17RE, IL1B, IL3, IL7R, IL9R, and EBI3, in a biological sample obtained from the subject, and
an apparatus as defined in claim 12.

15. Use of the kit as defined in claim 14.

16. The use as defined in claim 15 in a method of predicting a response of a prostate cancer subject to radiotherapy.

17. A method, comprising:
receiving a biological sample obtained from a prostate cancer subject,
using the kit as defined in claim 14 to determine a gene expression profile for each of three or more, for example, 3, 4, 5 or all, interleukin genes selected from the group consisting of: IL17RE, IL1B, IL3, IL7R, IL9R, and EBI3, in the biological sample obtained from the subject.

18. Use of a gene expression profile for each of three or more, for example, 3, 4, 5 or all, interleukin genes selected from the group consisting of: IL17RE, IL1B, IL3, IL7R, IL9R, and EBI3, in a method of predicting a response of a prostate cancer subject to radiotherapy, comprising:
determining, by a processor, the prediction of the radiotherapy response based on the gene expression profiles for the three or more interleukin genes, and
optionally, providing the prediction or a therapy recommendation based on the prediction to a medical caregiver or the subject.
